(19) United States
(12) Patent Application Publication
(10) Pub. No.: US 2007/0279217 A1
(43) Pub. Date: Dec. 6, 2007

(54) INTEGRATED MOBILE HEALTHCARE SYSTEM FOR CARDIAC CARE

(75) Inventors: Mahesh VENKATRAMAN, Palo Alto, CA (US); Surendar Magar, Dublin, CA (US)

Correspondence Address: SAWYER LAW GROUP LLP, P O BOX 51418, PALO ALTO, CA, 94303, US

(73) Assignee: H-Micro, Inc., Los Altos, CA (US)

(21) Appl. No.: 11/756,161

(22) Filed: May 31, 2007

Related U.S. Application Data
(60) Provisional application No. 60/810,742, filed on Jun. 1, 2006.

(51) Int. Cl. G08B 1/08; A61B 5/00

(52) U.S. Cl. 340/539.12; 600/300

(57) ABSTRACT

A distributed sensor based mobile/remote monitoring system for the management of various types of disease is disclosed. The system is capable of continuously monitoring a variety of parameters relating to the state of various diseases. The parameter monitoring can be continuous, periodic or episodic. A system to manage particular types of diseases can be defined by selecting appropriate parameters for that disease. A cardiac care product, based on the distributed sensor based mobile/remote monitoring system, is also disclosed. The product comprises a distributed sensor system including at least one patch for wirelessly monitoring a physiological parameter; and a biostrip for providing analysis of the blood of a person. The product further includes a mobile device for receiving signals from the distributed sensor system. The mobile device has one mechanism that includes a real-time wireless monitoring of the physiological parameter and a second mechanism that monitors the analysis of the blood.

### CROSS-REFERENCE TO RELATED APPLICATIONS

Under 35 U.S.C. 119, this application is a Non-Provisional application of U.S. Provisional Application No. 60/776,590, filed Feb. 24, 2006 and U.S. Provisional Application No. 60/810,742, filed Jun. 1, 2006, all of which are incorporated herein by reference.

### FIELD OF THE INVENTION

The present invention relates generally to cardiac health monitoring systems and more particularly to a health monitoring system that utilizes a medical signal processor with a wireless distributed sensor system.

### BACKGROUND OF THE INVENTION

Monitoring the health of people has always been important. As the population ages and more people advance in age, health monitoring systems become more significant to maintaining a healthy lifestyle and disease management. Mobile/remote health monitoring makes it easier and cost effective to monitor the health of vast populations. Wireless systems are the most desired approach to enable remote health monitoring. Therefore, a variety of wireless health monitoring systems have been introduced over the years.

Conventional wireless health monitoring systems are bulky, expensive, have inadequate wireless link reliability and have high power dissipation which severely limits their applications, particularly to monitor wide ranging physiological parameters in high volumes for large populations. Accordingly, what is desired is a mobile healthcare system that addresses the above-identified issues.

### SUMMARY OF THE INVENTION

A cardiac care product for mobile healthcare is disclosed. The product includes a distributed sensor system which comprises the distributed sensor system that includes at least two types of sensors resulting in dual-mode use of the system. In one mode, called continuous mode, the tiny wireless body sensors are either attached to the body or implanted within the body of a person to continuously monitor the physiological parameters (e.g. ECG signals) over longer periods of time. The second mode, called instantaneous mode, is supported by including sensors within the mobile device for instantaneous monitoring of certain physiological parameters (e.g. ECG signals, PT/INR analysis based on test strips).

The product further includes a mobile device for receiving signals from the distributed sensor system by using two types of mechanisms. The first mechanism supports receiving radio signals from the wireless body sensors for the continuous mode; and the second mechanism supports receiving signals from local wired sensors for the instantaneous mode. These two mechanisms are either built as an integral part of the mobile device or implemented via add-in cards (e.g. secure digital, SD, cards).

A specific product includes a mobile device with two add-in cards. One card carries the means to receive and process signals from wireless body sensors for continuous monitoring of various physiological parameters (e.g. ECG, EEG, EMG, physical activity). The second card supports the monitoring of blood chemistry related parameters (e.g. anticoagulation PT/INR, glucose) via the dry-chemistry based micro-fluidic blood test strips.

A distributed sensor based mobile/remote monitoring system for the management of various types of diseases is disclosed. The system is capable of continuously monitoring a variety of parameters relating to the state of various diseases. The parameter monitoring can be continuous, periodic or episodic. A system to manage a particular type of disease can be defined by selecting the appropriate parameters for that disease.

### BRIEF DESCRIPTION OF DRAWINGS

FIG. 1A is a block diagram of a first embodiment of a general architecture of a wireless health monitoring system in accordance with the present invention.

FIG. 1B is a block diagram of a second embodiment of a general architecture of a wireless health monitoring system in accordance with the present invention.

FIG. 2 illustrates examples of various sensors that can be included in a distributed sensor system.

FIG. 3 is a block diagram of a mobile cardiac care product in accordance with the present invention.

FIG. 4 is a block diagram of an implementation of a mobile device utilized with the cardiac care product of FIG. 3.

### DETAILED DESCRIPTION

The present invention relates generally to mobile health monitoring systems and more particularly to a health monitoring system that utilizes a medical signal processor with a wireless distributed sensor system. The following description is presented to enable one of ordinary skill in the art to make and use the invention and is provided in the context of a patent application and its requirements. Various modifications to the preferred embodiments and the generic principles and features described herein will be readily apparent to those skilled in the art. Thus, the present invention is not intended to be limited to the embodiments shown, but is to be accorded the widest scope consistent with the principles and features described herein.

To describe the feature of the mobile healthcare system in more detail, refer now to the following description in conjunction with the accompanying figures.

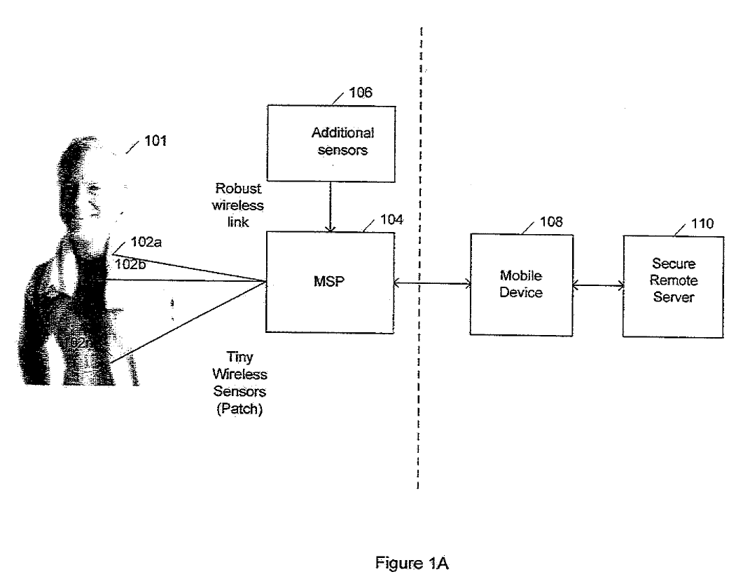

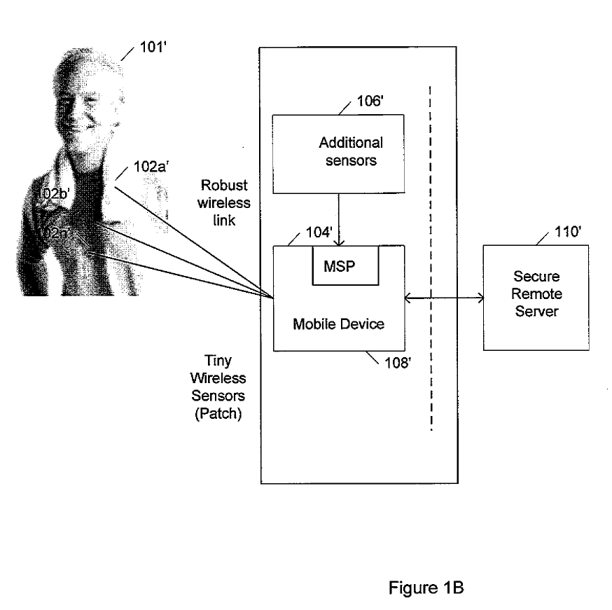

FIG. 1A is a first embodiment of a general architecture of a wireless mobile healthcare system 100 that serves as a baseline for the present invention. The system 100 is centered around a medical signal processor 104 that has a wireless distributed sensor system as its peripheral. The distributed sensor system includes a plurality of patches 102a-102n on a person 101. The patches 102a-102n can be internal to the body, coupled to the exterior of the body embedded in the garments or can be in close proximity of the body by some other means. The patches communicate wirelessly with MSP 104. The MSP 104 also includes its internal/local sensors 106, which can engage the body of the person, which are also part of the distributed sensor system. The medical signal processor (MSP) 104 in turn communicates with a mobile device 108. The mobile device 108 in turn communicates with a secure server 110 via a wireless or wired network. In this embodiment, the MSP 104 is a separate component from the mobile device 108. However, one of ordinary skill in the art readily recognizes that the MSP 104 could be incorporated into the mobile device as shown in FIG. 1B which is a second embodiment of the system 100′. The MSP 104 also includes sensors 106, which can engage the body of the person, which are also part of the distributed sensor system.

The mobile device 108 could be, for example, a cellular telephone, laptop, notebook, a smart phone, a PDA, a custom medical device or any mobile device which can communicate with the server over a network. Each component of the health monitoring system 100 will now be described in detail in conjunction with the accompanying figures.

### Medical Signal Processor

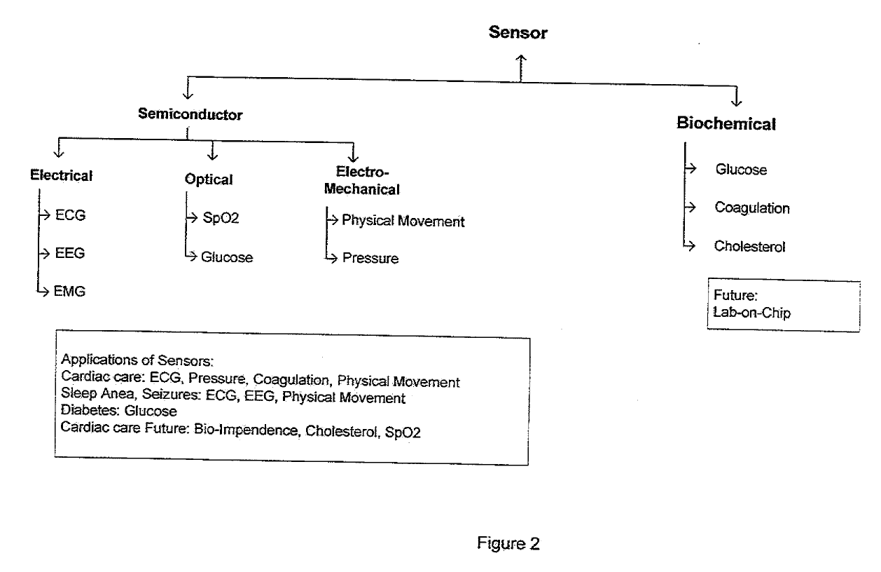

As discussed above, the medical signal processing system as shown in FIGS. 1A and 1B can include a variety of sensors—either directly integrated in the medical signal processor 104, or linked to the medical signal processor 104 via a wireless link as patches 102 on the body of a user. Examples of various sensors that can be included in the distributed sensor system are shown in FIG. 2. Out of these examples, certain sensors can be chosen for implementation as patches 102. Other sensors can be chosen for integration within the MSP 104. In this way, a variety of systems can be designed for the management of diseases, health and fitness, by choosing the sensors that monitor the appropriate parameters associated with target applications.

Modes of Operation: By using the distributed sensor system, the system of FIGS. 1A and 1B can monitor parameters in different modes. For example, by wearing patches on the body, the monitoring can be done in a continuous mode—data continuously flowing from sensors in to the mobile device to the secure server. Patches can also be used for periodic or episodic monitoring. In an instantaneous mode, monitoring is normally done by using the MSP 104 and sensors 106. For example, a cardiac rhythm can be directly monitored by pressing the MSP 104 against the body by using a built in ECG sensor. Another example of this stand-alone mode is glucose, cholesterol or blood anticoagulation PT/INR monitoring. A drop of blood is placed on a micro-fluidic test strip with dry chemical reagent to start a chemical reaction. The test strip is inserted into MSP 104 which can monitor desired blood parameter through an electrical or optical sensor built into it. The glucose, cholesterol or blood coagulation rate reading will be registered in the database on MSP 104 and/or mobile device 108 and/or the secure server 108.

### Wearable Wireless Patches 102

Patches 102 are integrated circuit technology driven miniature wireless devices that can be conveniently attached to the body. The patch 102 in a preferred embodiment has two main parts: sensor circuits, and a wireless radio core for the transmission of sensor data to other devices. In addition, it has a signal processor and power management circuits. In a preferred embodiment, a person can wear a patch 102 for several days for continuous monitoring without changing the battery.

### Mobile Device 108

The mobile device 108 could be, for example, a cellular telephone, laptop, notebook, a smart phone, a PDA, a custom medical device or any mobile device which can communicate with the server over a wide area network and/or Internet. The mobile device 108 can also be a regular cell phone handset, which has been modified to include the appropriate features and means to work with MSP 104. The mobile device 108 communicates with the MSP 104. In one embodiment, the MSP can be built within mobile device 108 as part of the mobile device design. In this mode, many internal functions of MSP can be implemented in software. In most cases, the MSP's radio system and sensor interfaces will remain intact in hardware.

### Secure Server 110

The secure server 110 receives data from distributed sensors over a cellular telephony network, any type of wide area network or Internet via MSP 104 and the mobile device 108. The server 110 further processes the received data from the mobile device and stores it in a secure location. The server 110 may also contain various types of software programs, including software to manage health information databases (such as electronic medical records, computerized purchase orders and computerized prescription systems). The secure server 110 may also have the middleware to process/link sensor data to such health information databases.

The data stored on the secure server 110 may be accessed by a healthcare provider, caregiver or patient via the Internet by using any type of terminal device such as computer, mobile device, cell phone, smart phone or personal data assistant (PDA).

The mobile healthcare system in accordance with the present invention supports many classes of sensors for physiological data collection, such as:

1. The health monitoring system supports many classes of sensors for physiological data collection, such as:
(a) Sensors (either patches 102 or sensors 106) contacting the body 101 through gels, etc.
(b) Patches 102 embedded within the body 101 through surgical procedures.
(c) Patches 102 probing the body 101 through micro-needle based skin punctures.
(d) Sensors in close proximity of the body 101, e.g., probing using a microwave or optical beam.
(e) Sensors embedded in the MSP 104 or mobile device 108 for periodic or occasional use.

2. The mobile healthcare system in accordance with the present invention can support one of these sensors and/or patches or multiple sensors and/or patches from multiple classes.

3. The MSP 104 has the ability to collect data in real time from many such sensors and/or patches and to apply a chosen algorithm to combine signals from various sensors and/or patches to determine or predict a physiological or disease state.

4. The MSP 104 can store data for local use and/or transmit in real time to a remote server for use by clinicians and other parties. If desired, some of the MSP 104 functions can be implemented on a remote sensor.

5. As stated above, one function of the MSP 104 is physiological data processing.

The health monitoring system in accordance with the present invention can be utilized in a variety of environments. One example is the cardiac disease management system. To describe the features of such a system refer now to the following description in conjunction with the accompanying figures.

### An Integrated Mobile Healthcare System for Cardiac Care

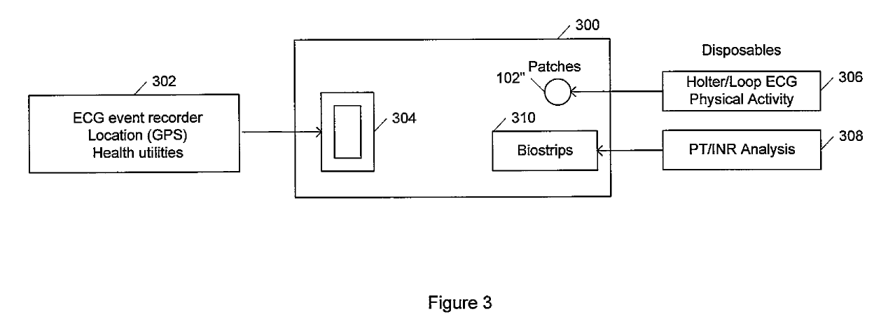

An embodiment of a cardiac disease care product in accordance with the present invention is described herein below. FIG. 3 is a block diagram of a cardiac care product in accordance with the present invention showing the overall functionality of the product. The cardiac care product includes a mobile device 304 which also contains MSP 104 described previously. The MSP 104 can have the needed functions such as wireless link, processor and storage to enable real-time wireless monitoring using patches 102. In addition, it can have a built-in ECG sensor to enable cardiac event monitoring based on using mobile device 304 in a stand-alone mode. The mobile device 304 can receive data from one or multiple wireless body sensors or patches 102. The mobile device 304 can also derive the value of certain blood chemistry related parameters by using its built-in electrical/optical sensors with the MSP 104 that can read biostrips 310. The biostrips 310 are basically micro-fluidic test strips that have dry chemicals to work with the blood samples mixed with them. The system can be used to realize a variety of procedures for cardiac care, some of which are as follows:
a. Ambulatory ECG: The patches 102 can have ECG (electrocardiogram) sensors to allow mobile device 304 to carry out a variety of ECG based cardiac procedures described in medical literature—e.g., holter monitoring, loop recording, event monitoring, cardiac rhythm monitoring, one-lead to twelve-lead monitoring. These ECG procedures can last from a few seconds to many days. The duration and configuration of ECG can be selected by the mobile device 304.
b. Pacemaker: Mobile device 304 can also wirelessly connect to a patch 102 which can be a pacemaker. The mobile device 304 can control a pacemaker as needed and also collect any needed data from it.
c. Pulse/Respiration: The pulse and respiration information can also be derived from an ECG signal received from a patch 102.
d. Physical activity: A patient's physical activity can be monitored by using accelerometer on patches 102. It allows for recording of patient movement along with ECG signals, thereby providing useful information for cardiac care.
e. Cardiac event monitoring: When needed, an instantaneous (short) ECG can be recorded by pressing the mobile device 304 on the chest without the use of any patches 102. This is possible due to the ECG sensor built within the mobile device as part of the MSP 104. This procedure of obtaining a short ECG record (a minute or so) is called cardiac event recording.
f. PT/INR analysis: PT/INR analysis showing blood anticoagulation rate is often used in the treatment of arrhythmias such as atrial fibrillation. The PT/INR measurement can be done by mobile device 304 by using an appropriate biostrip 310 with an appropriate dry chemical and by designing in an electrical or optical sensor within the mobile device 504 to sense blood anticoagulation rate.
g. Patient location: The system can also determine patient location by utilizing the GPS (global positioning system) built into mobile device 304.
h. Software utilities: A variety of software utilities can be designed into mobile device to help with cardiac care, such as medication schedules, clinical visit schedules, treatment schedules and on-line information sources.
i. Other functions using biostrips: Many times cardiac disease is associated with diabetes. The glucose test strips can be used by mobile device 304 to obtain blood glucose reading in the same manner as PT/INR analysis. A cholesterol measurement system can also be implemented in the same manner.
j. Other types of continuous monitoring: Desired sensors can be built into patches 102 to monitor any other parameter that can be helpful for cardiac care. For example, EEG and EMG monitoring can be helpful in certain situations relating to stroke that is commonly associated with cardiac diseases.

All these functions can be combined in a variety of ways to design an integrated mobile cardiac healthcare system with the desired features and format. Many of these functions can be integrated within a mobile device 104. Many of them can also be implemented by using add-in cards or dongles for a mobile device 104, as discussed below.

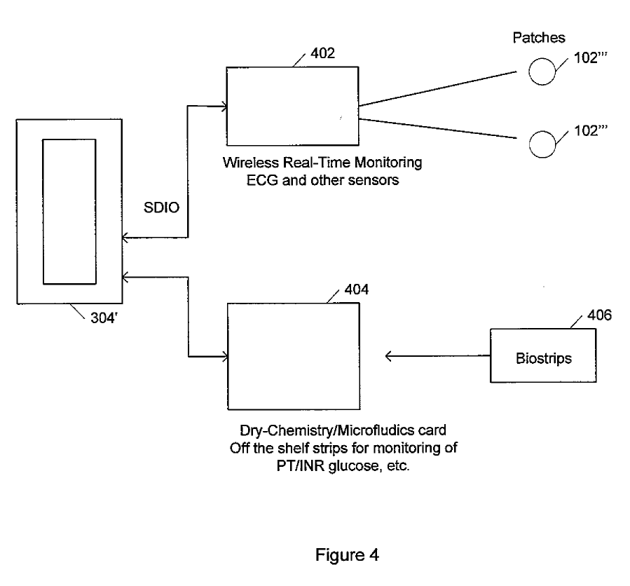

FIG. 4 is a block diagram of one implementation of a mobile cardiac healthcare system described in FIG. 3. It utilizes a mobile phone 304′ and two types of add-in cards for it (such as secure digital or SD format cards). One add-in card 402 contains MSP 104 to work with a variety of patches 102′″. The MSP 104′ has the needed functions such as wireless link, processor and storage to enable real-time wireless monitoring using patches 102′″. It can enable monitoring of functions such as ECG, physical activity, pulse, respiration, discussed above. The second add-in card 404 contains a slot and other needed means to accept a biostrip 406 and contains an electrical or optical sensor to read the biostrip 406. As discussed above, the biostrip 406 can have dry chemicals for blood analysis relating to functions such as anticoagulation (for PT/INR), glucose or cholesterol. The biostrips can be off-the-shelf test strips available from various vendors in the market. The GPS functionality can be embedded within the mobile device 304′ itself. A local ECG sensor can also be included with MSP 104 on the first add-in card. By using this sensor, when needed, an instantaneous (short) ECG can be recorded by pressing the mobile device 304 on the chest without the use of any patches 102. Various software utilities can be written for mobile device 304 for the patient's use as discussed above and for receiving the data from the MSP 104 on add-in cards. Many functions of the MSP 104 can also be implemented in software on the mobile device 304 for further data processing.

### CONCLUSION

An integrated mobile healthcare system for cardiac care is disclosed that uses a distributed sensor system and a mobile device. The system has a variety of means and modes to diagnose, treat and monitor the cardiac disease state. The system is highly integrated—basically a mobile device and a few tiny peripheral devices (wireless patches and micro-fluidic test strips). As described, such a compact mobile system can monitor a variety of crucial parameters relating to cardiac care—many modes of ECG, PT/INR, pacemaker, physical activity, EEG, EMG, glucose, cholesterol, etc. It will displace a suite of bulky and expensive systems. The utility of the system is further expanded via a built-in GPS device for patient location determination and a variety of software utilities for patient care. Such a mobile healthcare system opens a whole new way of diagnosing, treating and monitoring cardiac diseases.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. A cardiac care product comprising:
a distributed sensor system, the distributed sensor system comprising at least one patch for wirelessly monitoring a physiological parameter or physical activity; and a sensor for reading a biostrip for providing an analysis of the blood of a person; and
a mobile device for receiving signals from the distributed sensor system, wherein the mobile device includes a first mechanism to perform real-time wireless monitoring of a physiological parameter based on at least one wireless patch and a second mechanism to perform the analysis of the blood.

2. The cardiac care product of claim 1 wherein physiological signal is ECG; and biostrip/sensor is designed to perform blood anticoagulation PT/INR analysis for drug therapy for the treatment of arrhythmia.

3. The cardiac care product of claim 1 wherein mobile device also has a sensor to perform local ECG without the use of patches.

4. The cardiac care product of claim 1 wherein the distributed sensors monitors any or any combination of the following parameters: ECG signals; pulse and respiration; blood pressure; patient's physical movement; EEG signals; EMG signals and blood anticoagulation PT/INR analysis for drug therapy for the treatment of arrhythmia.

5. The cardiac care product of claim 1 wherein the analysis of the blood comprise any or any combination of the glucose monitoring analysis, cholestrol monitoring analysis and anticoagulation analysis.

6. The cardiac care product of claim 1 wherein the first mechanism monitors movement of the user via a patch.

7. The cardiac care product of claim 1 wherein the second mechanism comprises a microfluidics card.

8. The cardiac care product in claim 1 wherein each of the first and second mechanisms of the mobile device is built using add-in cards for the mobile device.

9. The cardiac care product of claim 8 wherein physiological signal is ECG; and the biostrip/sensor is designed to perform blood anticoagulation PT/INR analysis for drug therapy for the treatment of arrhythmia.

10. The cardiac care product of claim 9 wherein mobile device also has a sensor to perform local ECG without the use of patches.

11. The cardiac care product of claim 8 wherein the distributed sensors monitors any or any combination of the following parameters: ECG signals; pulse and respiration; blood pressure; patient's physical movement; EEG signals; EMG signals and blood anticoagulation PT/INR analysis for drug therapy for the treatment of arrhythmia.

12. The cardiac care product of claim 8 wherein the analysis of the blood comprise any or any combination of the glucose monitoring analysis, cholesterol momitoring analysis and anticoagulation analysis.

13. The cardiac care product of claim 8 wherein the first mechanism monitors movement of the user via a patch.

14. The cardiac care product of claim 8 wherein the second mechanism comprises a microfluidics card.
